(19) United States
(12) Patent Application Publication
(10) Pub. No.: US 2010/0040708 A1
(43) Pub. Date: Feb. 18, 2010

(54) HERBAL COMPOSITION

(76) Inventor: Vicki Boynton, Wilkeson, WA (US)

Correspondence Address: JAY M. SCHLOFF, 6960 Orchard Lake Road, Suite 315, West Bloomfield, MI, 48322, US

(21) Appl. No.: 12/509,531

(22) Filed: Jul. 27, 2009

Related U.S. Application Data
(60) Provisional application No. 61/088,459, filed on Aug. 13, 2008.

(51) Int. Cl. A61K 36/258; A61P 15/00

(52) U.S. Cl. 424/728

(57) ABSTRACT

An herbal composition for treating menopausal symptoms in a woman includes yarrow, damiana, skullcap, chaste tree berry, wild yam, corn silk, cramp bark, bloodroot, fenugreek, feverfew, cardamom, and panax ginseng. The herbal composition may either be in a liquid dosage form or in a solid dosage form. Further disclosed is a method for treating menopausal symptoms in a woman using the aforementioned herbal composition. The method includes orally administering the herbal composition, one to two times per day, to the woman.

### CROSS-REFERENCE TO RELATED APPLICATIONS

The present disclosure claims priority under 35 United States Code, Section 119 on the United States Provisional Patent Application numbered 61/088,459 filed on Aug. 13, 2008, the disclosure of which is incorporated by reference.

### FIELD OF THE DISCLOSURE

The present disclosure generally relates to herbal compositions, and, more particularly, to an herbal composition for treating menopausal symptoms in women.

### BACKGROUND OF THE DISCLOSURE

Menopause may be defined as a permanent cessation of menses or the end of menstrual cycles in women that generally occurs between the ages of forty and fifty-five. Menopause occurs due to a complex series of hormonal changes in a woman's body. More particularly, when a woman goes through menopause, there is a reduction in the levels of female hormones (including estrogen and progesterone.) In general, the onset of menopause is associated with a variety of symptoms (hereinafter referred to as “menopausal symptoms”) that often arise as a result of the reduced levels of estrogen hormone and progesterone hormone. The menopausal symptoms may vary amongst women but generally include mood swings, depression, aches, hot flashes, and the like. Further, the reduced levels of estrogen hormone and progesterone hormone may result in various physiological changes in a woman's body that may affect her health and well-being.

Conventional treatments for treating menopausal symptoms in a woman include the use of a hormone replacement therapy (HRT), for an indefinite time period. The HRT consists of administering either estrogen hormone, or estrogen hormone in combination with progesterone hormone to artificially boost hormone levels. However, it has been observed that the use of the HRT may have undesirable side effects, such as headache, diarrhea, nervousness, and the like, in women. Further, the use of the HRT is associated with risks of developing breast cancer and uterine cancer. In addition, the HRT provides a short-term relief from the menopausal symptoms. Moreover, the HRT is an expensive treatment.

Alternately, various pharmaceutical drugs have been used to alleviate discomfort caused by menopausal symptoms in women. Though the pharmaceutical drugs aid in treating menopausal symptoms, the pharmaceutical drugs are known to cause some mild or severe side-effects. Moreover, it has been observed that administration of the pharmaceutical drugs provides only short-term relief from the menopausal symptoms, which symptoms tend to recur as soon as the effect of the pharmaceutical drugs has passed.

Presently, various herbs including licorice root and angelica are also utilized for treating menopausal symptoms in women, as the use thereof is not associated with side effects found in conventional treatments. However, such herbs are capable of treating only a few of the menopausal symptoms, and accordingly, are incapable of providing relief from most or all of the menopausal symptoms. Further, many conventional herbal compositions have been prepared using processed forms of various herbs. However, these herbs are generally of an inorganic (synthetic) nature and tend to lose their effectiveness after such processing. In addition, the conventional herbal compositions may have unhealthy effects on women, as the conventional herbal compositions are processed synthetically.

Accordingly, there exists a need for an herbal composition that is capable of treating menopausal symptoms such as mood swings, depression, aches, hot flashes, and the like, in women, in an effective manner. Further, there exists a need for an herbal composition that is organic and healthy for women, for treating menopausal symptoms.

### SUMMARY OF THE DISCLOSURE

In view of the foregoing disadvantages inherent in the prior art, the general purpose of the present disclosure is to provide an herbal composition for treating menopausal symptoms in women, configured to include all the advantages of the prior art, and to overcome the drawbacks inherent therein.

Therefore, an object of the present disclosure is to provide an herbal composition that is capable of treating menopausal symptoms such as mood swings, depression, aches, hot flashes, and the like, in women, in an effective manner.

Another object of the present disclosure is to provide an herbal composition that is organic and healthy for women, for treating menopausal symptoms.

To achieve the above objects, in an aspect of the present disclosure, an herbal composition is provided for treating menopausal symptoms in a woman. The herbal composition includes yarrow, damiana, skullcap, chaste tree berry, wild yam, corn silk, cramp bark, bloodroot, fenugreek, feverfew, cardamom, and Panax ginseng. The herbal composition is orally administrable, and may be in either a liquid dosage form or a solid dosage form.

In another aspect of the present disclosure, a method of treating menopausal symptoms in a woman is provided. The method includes administering an herbal composition to the woman one to two times per day. The herbal composition includes yarrow, damiana, skullcap, chaste tree berry, wild yam, corn silk, cramp bark, bloodroot, fenugreek, feverfew, cardamom, and Panax ginseng. The herbal composition is administered orally with water.

These together with other aspects of the present disclosure, along with the various features of novelty that characterize the present disclosure, are pointed out with particularity in the claims annexed hereto and form a part of this present disclosure. For a better understanding of the present disclosure, its operating advantages, and the specific objects attained by its uses, reference should be made to the accompanying descriptive matter in which there are illustrated exemplary embodiments of the present disclosure.

### DETAILED DESCRIPTION OF THE DISCLOSURE

The exemplary embodiments described herein detail for illustrative purposes are subject to many variations. It should be emphasized, however, that the present disclosure is not limited to a particular herbal composition, as described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting. The use of terms, “including,” or “comprising,” and variations thereof herein is meant to encompass the items listed thereafter and equivalents thereof as well as additional items. Further, the terms “a” and “an” herein do not denote a limitation of quantity, but rather denote the presence of at least one of the referenced item.

The present disclosure relates to an herbal composition for treating symptoms of menopause (hereinafter referred to as “menopausal symptoms”) that include mood swings, depression, aches, hot flashes, and the like, in women. The term, “menopause” is defined as permanent cessation of menses or the end of menstrual cycles in women, and refers to peri-menopause, menopause, and post-menopause phases. Further, the term, “menopausal symptoms,” as used herein, refers to symptoms that are caused or exacerbated by reduction in levels of hormones, such as estrogen and progesterone, during the peri-menopause, the menopause, and the post-menopause phases. Furthermore, the terms, “treat,” “treating,” “treatment,” and variations thereof refer to alleviation, mitigation, or prevention of the menopausal symptoms, and include reduction in the severity or frequency of the menopausal symptoms.

The herbal composition of the present disclosure is a blend of herbs. Specifically, the herbal composition includes yarrow (Achiliea millefolium), damiana (Turnera diffusa), skullcap (Scutellaria lateriflora), chaste tree berry (Vitex agnus-castus), wild yam (Dioscorea villosa), corn silk (Zea mays), cramp bark (Viburnum opulus), bloodroot (Sanguinaria canadensis), fenugreek (Trigonella foenum-graecum), feverfew (Tanacetum parthenium), cardamom (Elettaria Cardamomum), and Panax ginseng. More particularly, the herbal composition includes about 0.70 grain of yarrow, about 0.70 grain of damiana, about 0.70 grain of skullcap, about 0.70 grain of chaste tree berry, about 0.70 grain of wild yam, about 0.70 grain of corn silk, about 0.70 grain of cramp bark, about 0.35 grain of bloodroot, about 0.70 grain of fenugreek, about 0.70 grain of feverfew, about 0.35 grain of cardamom, and about 0.35 grain of Panax ginseng.

Yarrow, as used in the herbal composition, is a medicinal herb and is capable of reducing problems such as restlessness and the like, in women during menopause. Further, yarrow possesses anti-catarrhal and anti-spasmodic activities, and is capable of reducing inflammation of ovaries. Furthermore, the aforementioned herb is aromatic, bitter, diaphoretic, haemostatic, hepatic, hypotensive, stomachic, and vulnerary in nature; and acts as a stimulant and a tonic. Damiana, as used in the herbal composition, acts as a tonic for the nervous system of a human body. Damiana is considered useful in the treatment of depression and anxiety in women. Further, damiana may be used for treating menopausal symptoms, and is particularly helpful for women with symptoms suggesting a deficiency of testosterone. Skullcap, as used in the herbal composition, is a powerful medicinal herb that is used as an analgesic, an anti-inflammatory agent, an anti-spasmodic agent, a mild astringent, an emmenagogue, a febrifuge, a hypnotic, a nervine, a sedative, and a strong tonic. More particularly, skullcap is useful for the treatment of anxiety and mood swings associated with hormonal changes during menopause.

Chaste tree berry, as used in the herbal composition, helps in increasing levels of progesterone hormone in women's body, regulating menstrual cycles, and treating menopausal symptoms. Wild yam, as used in the herbal composition, contains phytoprogesterone that helps in treating menopausal symptoms such as hot flashes, cramps, insomnia, and the like, in women. Further, the aforementioned herb may be used as an anti-inflammatory agent, an anti-bilious agent, an anti-emetic agent, an anti-spasmodic agent, and a cholagogue. Corn silk, as used in the herbal composition, reduces fluid retention, muscle cramps, and, pain and burning sensation associated with cystitis. Further, corn silk possesses anti-lithic and demulcent properties. Cramp bark, as used in the herbal composition, is an herb with anti-spasmodic, nervine, and sedative properties, and may be used for treating menopausal symptoms. Particularly, cramp bark is helpful for women with symptoms that suggest progesterone deficiencies.

Bloodroot, as used in the herbal composition, is a perennial plant and is known for providing relief from hot flashes during menopause. Fenugreek, as used in the herbal composition, is an herb with emollient and vulnerary properties. Further, fenugreek contains chemicals diosgenin and estrogenic isoflavones, which are similar to estrogen hormone. Accordingly, fenugreek helps in treating menopausal symptoms arising due to reduced levels of estrogen hormone. Feverfew, as used in the herbal composition, is helpful in relieving painful sensations associated with menopause. Specifically, feverfew is capable of treating hot flashes and other menopausal symptoms in women. Cardamom, as used in the herbal composition, acts as a natural diuretic for fluid retention, and helps ease menopausal symptoms. Further, cardamom acts as a carminative agent, and is aromatic in nature. Panax ginseng, as used in the herbal composition, helps in treating menopausal symptoms. Specifically, Panax ginseng helps in treating depression and in providing sense of well-being. Further, Panax ginseng helps preventing occurrence of mood swings and anxiety in postmenopausal women. More specifically, Panax ginseng acts as an adaptogen, which is capable of bringing abnormal physiology into balance.

The herbal composition of the present disclosure is an orally administrable medicament. In an embodiment of the present disclosure, the herbal composition may be in a liquid dosage form. Specifically, yarrow, damiana, skullcap, chaste tree berry, wild yam, corn silk, cramp bark, bloodroot, fenugreek, feverfew, cardamom, and Panax ginseng, may be dried, powdered, and then infused with grain alcohol for a specific time period. More specifically, about 0.70 grain of yarrow, about 0.70 grain of damiana, about 0.70 grain of skullcap, about 0.70 grain of chaste tree berry, about 0.70 grain of wild yam, about 0.70 grain of corn silk, about 0.70 grain of cramp bark, about 0.35 grain of bloodroot, about 0.70 grain of fenugreek, about 0.70 grain of feverfew, about 0.35 grain of cardamom, and about 0.35 grain of Panax ginseng may be dried and powdered, and then infused with grain alcohol for a time period of about four weeks to obtain the liquid dosage form. The liquid dosage form may thereafter be stored in a dropper bottle having a volume of about one ounce (oz.), for an easy withdrawal thereof. In use, a half teaspoon of the liquid dosage form of the herbal composition may be administered, one to two times per day to a woman showing menopausal symptoms. However, it may be evident to a person skilled in the art that the dose of the herbal composition, when prepared as the liquid dosage form, may vary depending upon the requirement, and specifically, depending upon severity and frequency of occurrence of menopausal symptoms in a woman. Further, the liquid dosage form may be administered with a glass of water.

In another embodiment of the present disclosure, the herbal composition may be in a solid dosage form. Specifically, the solid dosage form of the herbal composition is a capsule. More specifically, the capsule may be a “00” size capsule. However, it may be evident to a person skilled in the art that the herbal composition may be in any other solid dosage form such as a tablet, and the like.

The solid dosage form of the herbal composition may be prepared by drying and grinding each constituent of the herbal composition. Specifically, yarrow, damiana, skullcap, chaste tree berry, wild yam, corn silk, cramp bark, bloodroot, fenugreek, feverfew, cardamom, and Panax ginseng may be dried, ground, and then mixed to obtain a powdered form. More specifically, about 0.70 grain of yarrow, about 0.70 grain of damiana, about 0.70 grain of skullcap, about 0.70 grain of chaste tree berry, about 0.70 grain of wild yam, about 0.70 grain of corn silk, about 0.70 grain of cramp bark, about 0.35 grain of bloodroot, about 0.70 grain of fenugreek, about 0.70 grain of feverfew, about 0.35 grain of cardamom, and about 0.35 grain of Panax ginseng, may be dried, ground, and mixed to obtain a powdered form of the herbal composition.

Thereafter, the powdered form of the herbal composition may be incorporated into a size “00” capsule. Moreover, each capsule of the herbal composition may contain a half-grain constituted from the mixture of the aforementioned herbs when used in the above specified quantities. In use, the solid dosage form of the herbal composition may be administered one to two times per day, with a glass of water, to a woman showing menopausal symptoms. Specifically, a woman showing menopausal symptoms may ingest either one capsule or two capsules per day. However, it may be evident to a person skilled in the art that the dose of the herbal composition, when prepared in the solid dosage form, may vary depending upon the requirement, and specifically, depending upon severity and frequency of occurrence of menopausal symptoms in a woman. Further, it may also be evident to a person skilled in the art that the capsule may be a size “0” capsule, a size “000” capsule, and the like.

Furthermore, the herbal composition, whether prepared in the solid dosage form or the liquid dosage form, may exhibit a pale green-brown color due to the use of herbs for preparation thereof.

The herbal composition of the present disclosure acts as an herbal supplement for treating menopausal symptoms such as mood swings, depression, aches, hot flashes, and the like, in a woman. Specifically, the herbal composition possesses analgesic property, and accordingly, the herbal composition helps in reducing pain. Further, the herbal composition is anti-catarrhal in nature, and accordingly, the herbal composition helps in removing excess catarrhal build-ups from a woman's body. The excess catarrhal build-ups may occur in either sinus area or in other parts of the woman's body. Furthermore, the herbal composition possesses an anti-bilious property, and accordingly, the herbal composition is capable of removing excess bile from the woman's body. The herbal composition also possesses anti-emetic properties, as the herbal composition is capable of preventing nausea and vomiting.

The herbal composition serves as an anti-inflammatory agent that helps the woman's body to combat inflammations, and an anti-lithic agent that prevents the formation of stones or gravel in the urinary system and aids in removal of pre-formed stones or gravel from the woman's body. In addition, the herbal composition acts as an anti-spasmodic agent that prevents or eases spasms or cramps. Further, the herbal composition is aromatic in nature and has a strong and pleasant odor. The herbal composition is also capable of stimulating the digestive system of the woman. The herbal composition of the present disclosure acts as an astringent, and accordingly, the herbal composition is capable of contracting tissues by precipitating proteins and is capable of reducing secretions and discharges from/within the woman's body. In addition, most of the herbs included in the herbal composition are bitter to taste, and are known to act as stimulant tonics for the digestive system. Specifically, taste-initiated reflex activity by taste buds allows for stimulatory action of the herbs on the digestive system of the woman.

Further, the herbal composition is carminative, i.e., rich in volitive oils, and accordingly, the herbal composition is capable of stimulating peristaltic activity of the digestive system and relaxing the stomach, in order to support digestion. Moreover, the herbal composition acts as a cholagogue, as the herbal composition is capable of stimulating the release and secretion of bile from the gallbladder. Accordingly, the herbal composition exhibits a laxative effect. In addition, the herbal composition acts as a demulcent, as the herbal composition is rich in mucilage and is capable of soothing and protecting irritated or inflamed internal tissues. Also, the herbal composition is diaphoretic, and accordingly, the herbal composition aids in eliminating toxins from skin while promoting perspiration. Furthermore, the herbal composition acts as a diuretic agent that increases the secretion and elimination of urine from the woman's body. The herbal composition disclosed herein also acts as an emmenagogue, as the herbal composition is capable of stimulating and normalizing menstrual flow during menses.

The herbal composition of the present disclosure also serves as an emollient that softens, soothes, and protects the skin. Further, the herbal composition aids in toning and strengthening of the liver, and in increasing the flow of bile therefrom. The herbal composition is hypnotic and soporific, as the herbal composition is capable of inducing sleep. The herbal composition acts as a nervine that aids in toning and strengthening of the nervous system of the woman. The herbal composition further acts as a sedative, as the herbal composition is capable of relaxing the nervous system while reducing stress and nervousness throughout the woman's body. Additionally, the herbal composition acts as a stimulant that enhances and enlivens the physiological functions of the woman's body; a tonic that strengthens and enlivens either specific organs or the whole body; and as a vulnerary that aids in healing of wounds and cuts.

It should be understood that the aforementioned properties of the herbal composition of the present disclosure are attributed by one or more of the herbs that constitute the herbal composition.

In another aspect of the present disclosure, a method of treating menopausal symptoms in a woman is disclosed. The method includes administering the herbal composition one to two times per day, to the woman. As described above, the herbal composition includes yarrow, damiana, skullcap, chaste tree berry, wild yam, corn silk, cramp bark, bloodroot, fenugreek, feverfew, cardamom, and Panax ginseng. Specifically, the herbal composition includes about 0.70 grain of yarrow, about 0.70 grain of damiana, about 0.70 grain of skullcap, about 0.70 grain of chaste tree berry, about 0.70 grain of wild yam, about 0.70 grain of corn silk, about 0.70 grain of cramp bark, about 0.35 grain of bloodroot, about 0.70 grain of fenugreek, about 0.70 grain of feverfew, about 0.35 grain of cardamom, and about 0.35 grain of Panax ginseng. Further, the herbal composition may be administered orally with water. The herbal composition used for treating menopausal symptoms in a woman is explained above in detail, and accordingly, a description thereof is avoided herein for the sake of brevity.

As disclosed herein, the present disclosure provides an herbal composition for effectively treating menopausal symptoms such as mood swings, depression, aches, hot flashes, and the like, in women. Further, the herbal composition provides an organic and healthy way for treating the menopausal symptoms, as the herbal composition is a blend of fresh, dried, powdered, and unprocessed herbs, and is capable of retaining effective actions of the herbs constituting the herbal composition. Moreover, the herbal composition is free from any pharmaceutical drug that may have one or more side effects on women post-administration.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, and to thereby enable others skilled in the art to best utilize the present disclosure and various embodiments with various modifications as are suited to the particular use contemplated. It is understood that various omissions and substitutions of equivalents are contemplated as circumstances may suggest or render expedient, but such omissions and substitutions are intended to cover the application or implementation without departing from the spirit or scope of the claims of the present disclosure.

What is claimed is:

1. An herbal composition for treating menopausal symptoms in a woman, the herbal composition comprising:
yarrow;
damiana;
skullcap;
chaste tree berry;
wild yam;
corn silk;
cramp bark;
bloodroot;
fenugreek;
feverfew;
cardamom; and
Panax ginseng.

2. The herbal composition of claim 1, wherein the herbal composition comprises,
about 0.70 grain of yarrow,
about 0.70 grain of damiana,
about 0.70 grain of skullcap,
about 0.70 grain of chaste tree berry,
about 0.70 grain of wild yam,
about 0.70 grain of corn silk,
about 0.70 grain of cramp bark,
about 0.35 grain of bloodroot,
about 0.70 grain of fenugreek,
about 0.70 grain of feverfew,
about 0.35 grain of cardamom, and
about 0.35 grain of Panax ginseng.

3. The herbal composition of claim 1 is an orally administrable medicament.

4. The herbal composition of claim 3, wherein the herbal composition is in a liquid dosage form.

5. The herbal composition of claim 3, wherein the herbal composition is in a solid dosage form.

6. The herbal composition of claim 5, wherein the solid dosage form is a capsule.

7. The herbal composition of claim 6, wherein the capsule is a size “00” capsule.

8. A method of treating menopausal symptoms in a woman, the method comprising administering to the woman an herbal composition one to two times per day, the herbal composition comprising yarrow, damiana, skullcap, chaste tree berry, wild yam, corn silk, cramp bark, bloodroot, fenugreek, feverfew, cardamom, and Panax ginseng.

9. The method of claim 8, wherein the herbal composition comprises about 0.70 grain of yarrow, about 0.70 grain of damiana, about 0.70 grain of skullcap, about 0.70 grain of chaste tree berry, about 0.70 grain of wild yam, about 0.70 grain of corn silk, about 0.70 grain of cramp bark, about 0.35 grain of bloodroot, about 0.70 grain of fenugreek, about 0.70 grain of feverfew, about 0.35 grain of cardamom, and about 0.35 grain of Panax ginseng.

10. The method of claim 8, wherein the herbal composition is administered orally with water.

11. The method of claim 8, wherein the herbal composition is in a liquid dosage form.

12. The method of claim 8, wherein the herbal composition is in a solid dosage form.

13. The method of claim 12, wherein the solid dosage form is a capsule.

14. The method of claim 13, wherein the capsule is a size “00” capsule.
